(19) United States
(12) Patent Application Publication
(10) Pub. No.: US 2021/0407649 A1
(43) Pub. Date: Dec. 30, 2021

(54) THERAPY PLATFORM AND METHOD OF USE

(71) Applicant: Omada Health, Inc., San Francisco, CA (US)

(72) Inventors: Sean Patrick Duffy, San Francisco, CA (US); Andrew Paul DiMichele, San Francisco, CA (US); Adrian Benton James, San Francisco, CA (US)

(21) Appl. No.: 17/459,396

(22) Filed: Aug. 27, 2021

Related U.S. Application Data
(63) Continuation-in-part of application No. 15/667,218, filed on Aug. 2, 2017, now abandoned.
Application No. 15/667,218 is a continuation-in-part of application No. 14/180,205, filed on Feb. 13, 2014, now abandoned.
Application No. 14/180,205 is a continuation-in-part of application No. 13/668,644, filed on Nov. 5, 2012, now abandoned.
Application No. 15/667,218 is a continuation-in-part of application No. 14/190,017, filed on Feb. 25, 2014, now abandoned.
Application No. 14/190,017 is a continuation-in-part of application No. 13/668,644, filed on Nov. 5, 2012, now abandoned.
Application No. 15/667,218 is a continuation-in-part of application No. 14/245,961, filed on Apr. 4, 2014, now abandoned.
Application No. 14/245,961 is a continuation-in-part of application No. 13/668,644, filed on Nov. 5, 2012, now abandoned.
(60) Provisional application No. 61/555,455, filed on Nov. 3, 2011.
(60) Provisional application No. 61/555,455, filed on Nov. 3, 2011.
(60) Provisional application No. 61/555,455, filed on Nov. 3, 2011.

(51) Int. Cl. G16H 20/30; G16H 10/60; G16H 50/20; A61B 5/00; A61B 5/11

(52) U.S. Cl. CPC G16H 20/30; G16H 10/60; G16H 50/20; A61B 2562/0219; A61B 5/1118; A61B 5/4848; A61B 5/4836; A61B 5/0002

(57) ABSTRACT

Embodiments of a system and/or method can include: a set of weight sensor subsystems associated with the set of users, wherein a weight sensor subsystem of the set of weight sensor subsystems comprises a weight sensor operable to collect a weight dataset for a user, wherein the weight dataset is associated with a physical activity characteristic of the user, and a wireless communication module operable to transmit the weight dataset; and a medical improvement subsystem wirelessly connectable to the set of weight sensor subsystems, wherein the medical improvement subsystem is operable to: assign the user to a user subgroup based on the physical activity characteristic of the user; determine a physical activity metric based on the weight dataset; and promote a therapeutic intervention to the user based on the physical activity metric, where the therapeutic intervention is operable to improve the status of the first user.

### CROSS-REFERENCE TO RELATED APPLICATIONS

This application is a continuation-in-part of U.S. application Ser. No. 15/667,218 filed 2 Aug. 2017, which is a continuation-in-part of U.S. application Ser. No. 14/180,205 filed 13 Feb. 2014, U.S. application Ser. No. 14/190,017 filed 25 Feb. 2014, and U.S. application Ser. No. 14/245,961 filed 4 Apr. 2014, each of which is a continuation-in-part of U.S. application Ser. No. 13/668,644 filed 5 Nov. 2012, which claims the benefit of U.S. Provisional App. No. 61/555,455 filed 3 Nov. 2011, each of which are incorporated in their entirety by this reference.

### TECHNICAL FIELD

This invention relates generally to the medical field, and more specifically to an improved method for supporting a health regimen in the medical field.

### BACKGROUND

It is well known that people with excess body weight (e.g. body fat) have increased risk of health problems, such as diabetes and cardiovascular disease. Medical professionals generally advise overweight or obese patients to lower their risk of health complications by losing excess weight. For example, people with pre-diabetes (a condition in which glucose levels are higher than normal but are not high enough for a diagnosis of diabetes) can delay or lower their risk of developing diabetes by losing a modest amount of weight through dietary changes and increased physical activity. However, despite general guidelines such as improved diet or increased exercise, it may be difficult for many to effectively lose weight. Generic guidelines may not be suitable or useful for certain individuals, and many may not have access to personal nutritionists or trainers. Drastic lifestyle changes are often difficult to implement, and may contribute to lost motivation that hampers effective weight loss. Thus, there is a need in the medical field to create an improved method and user interface for supporting a health regimen. This technology provides such an improved method and system.

### BRIEF DESCRIPTION OF THE FIGURES

FIGS. 1 and 2 are schematics of an embodiment of a method for supporting a health regimen of a preferred embodiment;

FIG. 3 is a schematic of an example of filtering measurement data in the method of a preferred embodiment;

FIGS. 4A and 4B are examples of determining physical activity metrics associated with the body metric measurement data of a participant and of a matched group;

FIG. 5A depicts an embodiment of a user interface for supporting a health regimen;

FIG. 5B is an example of a home page in an example embodiment of a user interface for supporting a health regimen;

FIG. 6 is an example of a profile page in an example embodiment of a user interface for supporting a health regimen;

FIG. 7 is an example of a progress page in an example embodiment of a user interface for supporting a health regimen;

FIG. 8 is an example group page in an example embodiment of a user interface for supporting a health regimen;

FIGS. 9A and 9B are example communications between participants in an example embodiment of a user interface including a message client;

FIG. 10 is an example curriculum page in an example embodiment of a user interface for supporting a health regimen;

FIG. 11 is an example communication between a facilitator and a participant in an example embodiment of a user interface for supporting a health regimen;

FIG. 12 is a second example of a profile page in a second example embodiment of a user interface for supporting a health regimen;

FIG. 13 is a second example of a group page in a second example embodiment of a user interface for supporting a health regimen;

FIG. 14 is a second example of a curriculum page in a second example embodiment of a user interface for supporting a health regimen;

FIG. 15 is a sample health regimen curriculum scheme based on a diabetes prevention program;

FIG. 16 depicts an embodiment of a system for supporting a health regimen;

FIG. 17 is schematic diagram of an exemplary architecture that includes a health program tracking system for practicing aspects of the present technology;

FIG. 18 illustrates an exemplary computing system that may be used to implement embodiments according to the present technology;

FIG. 19 is a GUI that includes a first visual display for a first participant that is indicative of the current completion percentage for the task of a sub-program; and

FIG. 20 is a GUI that illustrates visual indicators that illustrate when a participant has successfully completed a task.

### DESCRIPTION OF THE EMBODIMENTS

The following description of embodiments of the invention is not intended to limit the invention to these embodiments, but rather to enable any person skilled in the art to make and use this invention.

### 1. Overview.

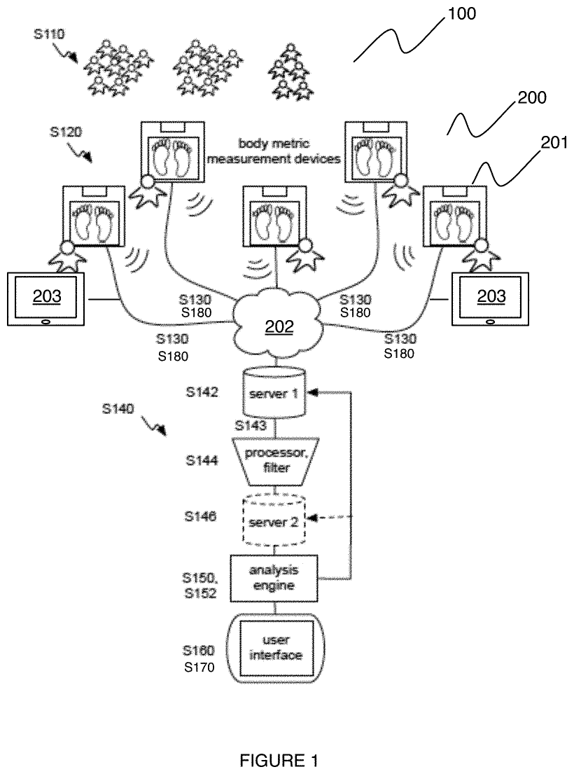

As shown in FIG. 1, embodiments of a system 200 for improving a status (e.g., condition) of a first user from a set of users (e.g., through improved distribution of functionality across the system 200, etc.) can include a set of weight sensor subsystems 201 associated with the set of users (e.g., set of humans), where a weight sensor subsystem of the set of weight sensor subsystems 201 includes a weight sensor operable to collect a weight dataset for a user, where the weight dataset is associated with a physical activity characteristic of the user, and a wireless communication module operable to transmit the weight dataset; and a medical improvement subsystem 202 wirelessly connectable to the set of weight sensor subsystems 201, where the medical improvement subsystem 202 is operable to: assign the user to a user subgroup (e.g., matched group) based on the physical activity characteristic of the user (e.g., as in Block S110; where the user subgroup is operable to improve processing of the weight dataset by the medical improvement subsystem 202; etc.); receive the weight dataset from the weight sensor subsystem 201 (e.g., as in Block S130); determine a physical activity metric based on the weight dataset (e.g., as in Block S150); and promote a therapeutic intervention to the user based on the physical activity metric (e.g., as in Block S160), where the therapeutic intervention is operable to improve the status of the first user. In embodiments of the system 200, the system 200 can additionally or alternatively include: a set of motion sensor subsystems 203 associated with the set of users; a user interface operable to improve display of physical activity metrics; and/or any other suitable components.

Embodiments of the system and/or method can function to improve a network of non-generalized system components (e.g., including a remote medical improvement system server 202, wireless weight sensor subsystems 201, wireless motion sensor subsystems 203, etc.) in order to improve processing of body metric measurement data in characterizing and improving user statuses (e.g., associated with diabetes) through therapeutic interventions personalized to the users and associated user subgroups. However, the system 200 and/or method 100 can possess any suitable functionality

The system 200 and/or method 100 are preferably used to facilitate a social environment in which the participants interact with a facilitator and/or one another to more effectively follow a health regimen. A facilitator leading the matched group and/or the participants in the matched group may provide feedback and support tailored to the matched group overall and/or to individual participants in the matched group. In one preferred embodiment, the system 200 and/or method 100 is used to help guide participants diagnosed with prediabetes to lose weight to reduce their risk of developing diabetes. In particular, the system 200 and/or method may be used to guide participants through the steps outlined in the Diabetes Prevention Program (a research study funded by the National Institute of Diabetes and Digestive and Kidney Diseases). The National Diabetes Prevention Program core curriculum, core session handouts, post-core curriculum, post-core session handouts, and additional materials (National Center for Chronic Disease Prevention and Health Promotion, Diabetes Training and Technical Assistance Center at the Rollins School of Public Health, Emory University) are incorporated herein by reference. In another embodiment, the system 200 and/or method 100 is used to help guide participants diagnosed with obesity to lose weight through an exercise and/or diet regimen. Furthermore, in alternative embodiments the system 200 and/or method 100 may be used to support health regimens regarding other body metrics, such as BMI, body fat percentage, blood pressure, cholesterol, or other suitable measurements. In variations of the embodiments, the system 200 and/or method 100 may be used in a group, support-oriented setting to monitor weight loss or gain in other applications, such as to monitor rapid weight gain indicative of swelling after a diagnosis of congestive heart failure, to monitor unintended weight loss suggestive of paraneoplastic syndrome after a diagnosis of cancer (e.g., prostate or lung cancer), to monitor weight fluctuations after diagnosis of hyper- or hypothyroidism or hyper- or hypoadrenalism (which may indicate, for example, medication dosing errors or changes in the endocrine defect), or to monitor weight trends after diagnosis of eating disorders such as anorexia. In some alternative variations of the embodiments, the system 200 and/or method 100 may omit grouping the participants into at least one matched group, such that trends and feedback are determined on an individual basis only.

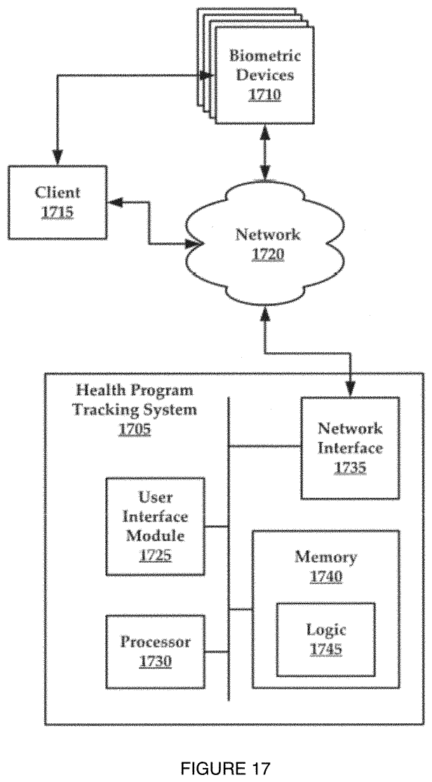

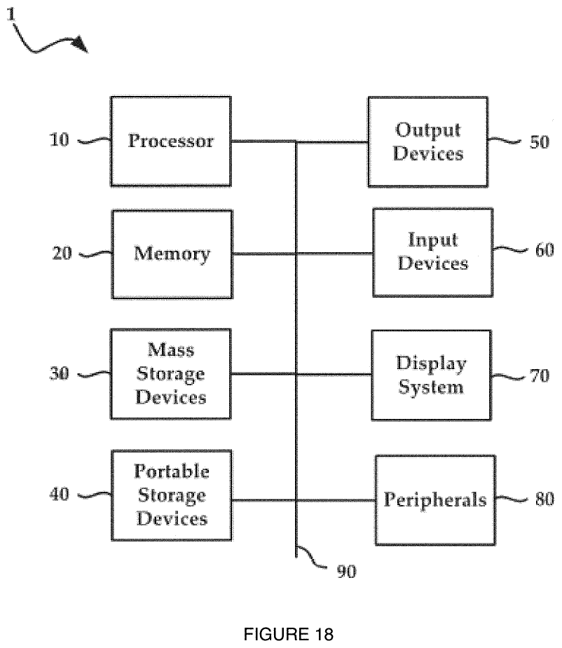

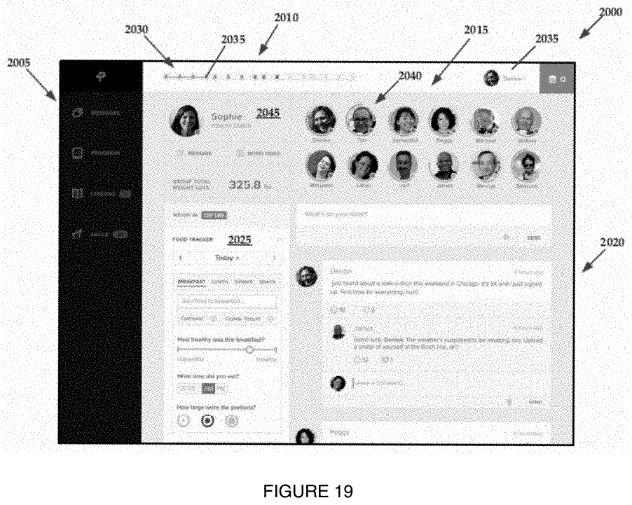

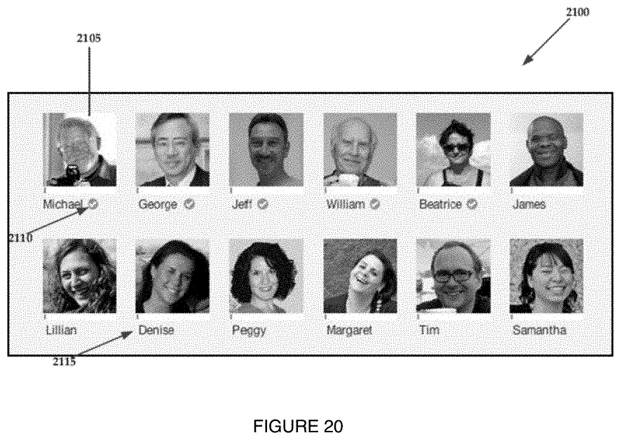

One or more instances of the method 100 and/or processes described herein can be performed asynchronously (e.g., sequentially), concurrently (e.g., in parallel such as through aggregate processing a plurality of weight and motion datasets from a user subgroup; concurrently on different threads for parallel computing to improve medical improvement system 202 processing ability; etc.), in temporal relation to a trigger event, and/or in any other suitable order at any suitable time and frequency by and/or using one or more instances of the system 200, elements, and/or entities described herein. Additionally or alternatively the method 100 and/or system 200 can be configured in any manner analogous to U.S. application Ser. No. 14/180,205 filed 13 Feb. 2014, U.S. application Ser. No. 14/190,017 filed 25 Feb. 2014, and U.S. application Ser. No. 14/245,961 filed 4 Apr. 2014 (e.g., such as to system components described in FIGS. 17-20), each of which are herein incorporated in their entirety by this reference. However, the method 100 and/or system 200 can be configured in any suitable manner.

### 2. Benefits.

The system 200 and/or method can confer several benefits over conventional methodologies. First, conventional approaches can suffer from inability to track user progress over time, leading to insufficient body metric measurement data for accurately characterizing user status and supporting users with personalized therapeutic interventions over time. Second, conventional health regimens can be associated with poor user adherence, which can be attributed at least in part to lack of sufficient peer support and/or facilitator support. Third, conventional approaches can fail to provide a digital network tailored to wirelessly connecting non-generalized body metric measurement devices for seamlessly collecting and processing body metric measurement data in providing physical activity insights (e.g., as part of physical activity metrics) and/or support to users. Examples of the system 200 and the method 100 can confer technologically-rooted solutions to at least the challenges described above.

First, the technology can confer improvements in the computational processing capabilities of components of the system 200. For example, the technology can computationally determine user subgroups (e.g., based on demographics, physical activity characteristics, etc.) including users who progress through a group program together (e.g., to enable peer support for a health regimen), where the classification of users into subgroups can improve data storage, retrieval, and analysis of body metric measurement data collected for the users. In specific examples, collected body metric measurement data for users from a user subgroup can be stored in association with a user subgroup identifier; retrieved (e.g., in aggregate; for a subset of users of the user subgroup; etc.) based on user subgroup identifier (e.g., to improve retrieval speed for user subgroup-associated data); and processed in relation to the user subgroup to improve the accuracy of characterization and treatment of users within the user subgroup. In another example, the technology can improve the application of weight sensors, inertial sensors, and/or other suitable activity-related sensors as tools, such as through providing an expansive digital network wirelessly connecting medical improvement systems (e.g., remote servers), weight sensor subsystems, and/or motion sensor subsystems across populations of users to extend the applicability of activity-related sensors (e.g., biometric sensors, optical sensors, etc.) to digital environments including peer support (e.g., through user subgroups) and/or facilitator support (e.g., through enabling wireless communication between users and facilitators) for health regimens. As such, the technology can amount to an inventive distribution of functionality across a network for improving data aggregation, data processing, and/or user experience, such as through distributing data collection and automatic transmission functionality across a plurality of wireless weight sensor subsystems and/or wireless motion sensor subsystems assigned to (e.g., linked with corresponding user accounts) and provided to users within a user subgroup; and distributing data storage, retrieval, and/or analysis functionality and/or therapeutic intervention provision functionality to the medical improvement system for optimizing user progress tracking and user status improvement. The technology can thus provide a full-stack approach leading to improvements in healthcare costs and disease prevention (e.g., diabetes prevention).

Second, the technology can provide technical solutions necessarily rooted in computer technology (e.g., leveraging a medical improvement network including connected weight sensor subsystems with weight sensors; connected motion sensor subsystems with inertial sensors; remote servers; and/or other suitable components to enable a user subgroup to progress through a digitally administered program together; etc.) to overcome issues specifically arising with computer technology (e.g., enabling a digital network of non-generalized devices such as activity-related sensors; digitally providing peer support and/or facilitator support for users remote from each other; computationally determining and providing physical activity metrics and/or therapeutic interventions tailored for optimizing user adherence and improvement; etc.). In an example, the technology can apply computer-implemented rules (e.g., feature engineering rules for processing body metric measurement data into an operable form for extracting relevant physical activity metrics and/or therapeutic interventions in relation to users and corresponding user subgroups; etc.) in conferring improvements to the computer-related technical field of digital healthcare.

Third, the technology can improve the technical fields of at least computer networks, body metric measurement devices, digital healthcare, digital communication (e.g., between users, facilitators, etc.), and/or other relevant fields. The technology can continuously collect and utilize specialized datasets unique to network-enabled, non-generalized body metric measurement devices in order to better characterize and/or treat user statuses. Further, the technology can take advantage of such devices and datasets to better improve the understanding of correlations between user behaviors, physical activity metrics, and appropriate therapeutic interventions.

Fourth, the technology can transform entities (e.g., users, body metric measurement devices, specialized datasets collected from activity-related sensors, etc.) into different states or things. For example, the technology can identify therapeutic interventions to promote to a user for improving user statuses (e.g., in relation to weight, cardiovascular health, diabetes, etc.) thereby transforming the health of the user. In another example, the technology can activate, control, and/or otherwise interact with body metric measurement devices to promote therapeutic interventions (e.g., by generating control instructions for the device to execute), thereby transforming the physical activity-related devices.

Fifth, the technology can confer improvements in computer-related technology by facilitating performance of functions not previously performable, such as computer network-related functions that the technology can leverage to enable functionality of the medical improvement network of body metric measurement devices and remote medical improvement systems.

Sixth, the technology can improve the accuracy of body metric data measurements, physical activity metrics, and therapeutic interventions. In an example and as discussed in further detail below, this technology can apply computer-implemented filtering rules to flag measurements based on measurement trends. This technology can additionally improve the accuracy of a user's physical activity metric(s) and therapeutic intervention(s) by leveraging the user subgroup to provide additional data, data context, and network information.

The technology can, however, provide any other suitable benefit(s) in the context of using non-generalized computer-related systems for supporting health regimens.

### 3.1 System—Weight Sensor Subsystem.

Weight sensor subsystems 201 of the system 200 function to collect and/or transmit weight datasets for a set of users (e.g., for a user subgroup). A weight sensor subsystem 201 preferably includes one or more weight sensors, one or more communication modules (e.g., a wireless communication module operable to transmit weight datasets; to receive over-the-air updates to firmware and/or software from the medical improvement system 202; etc.), and/or any other suitable components. A set of weight sensor subsystems 201 is preferably associated with a set of users (e.g., a different weight sensor subsystem distributed and assigned to each user of a user subgroup, etc.). For example, a weight sensor subsystem 201 can be automatically linked to the user account prior to distribution of the weight sensor subsystem 201 to the first human (e.g., where the medical improvement system 202 stores a weight sensor subsystem identifier in association with a user account identifying the user who is assigned the weight sensor subsystem 201; where the medical improvement system 202 can automatically store the weight dataset in association with the user account and/or other suitable data such as a user subgroup identifier in response to receiving the weight dataset from the weight sensor subsystem 201; etc.). Additionally or alternatively, any other suitable body metric measurement devices (e.g., motion sensor subsystems 203) can be automatically linked to any one or more users. However, body metric measurement devices can be associated with users in any suitable manner.

A weight sensor of a weight sensor subsystem 201 preferably samples weight datasets describing a body weight of a user, but weight datasets can describe and/or be processed to describe any suitable weight-related parameter. Additionally or alternatively, weight sensor subsystem 201 and/or other body metric measurement devices can include any suitable sensors. However, weight sensors and/or weight sensor subsystems 201 can be configured in any suitable manner.

### 3.2 System—Medical Improvement System.

The medical improvement system 202 (e.g., medical improvement subsystem) functions to perform one or more portions of the method 100. For example, medical improvement system 202 can be operable to: assign a set of users to a user subgroup (e.g., based on a shared physical activity feature from a set of physical activity features); collect (e.g., wirelessly) body metric measurement data (e.g., weight datasets, motion datasets, etc.); store the body metric measurement data (e.g., in association with user subgroups, user accounts, therapeutic interventions administered to the user at a time period associated with the body metric measurement data, etc.); retrieve the body metric measurement data (e.g., based on the human subgroup); determine physical activity metrics for the users (e.g., based on the body metric measurement data, etc.); determine a therapeutic intervention based on the physical activity metrics; and/or promote the therapeutic intervention to a user. The medical improvement system 202 can additionally or alternatively function to facilitate user progress through one or more group programs, such as those analogous to U.S. application Ser. No. 14/190,017 filed 25 Feb. 2014, which is incorporated in its entirety by this reference. However, the medical improvement system 202 can have any suitable functionality.

The medical improvement subsystem 202 is preferably wirelessly connectable (e.g., through a cellular network; WiFi, etc.) to any suitable body metric measurement devices, but can be connected to any suitable component of the medical improvement network in any suitable manner. The medical improvement subsystem 202 preferably includes one or more remote computing systems (e.g., a server, at least one networked computing system, stateless, stateful), but can additionally or alternatively include a local computing system, a device associated with a user and/or facilitator, a treatment system, databases (e.g., for body metric measurement data, physical activity metrics, therapeutic interventions, identifiers, user interface components, etc.), and/or any other suitable components.

In variations, the medical improvement system 202 can additionally or alternatively include one or more treatment systems, which can function to promote therapeutic interventions. Additionally or alternatively, treatment systems can function to collect body metric measurement data. In an example, a biometric subsystem (e.g., biometric device) can be operable to collect blood sugar values, heart beat values, blood pressure, temperature, weight, body mass index values, body fat percentage, hydration, and/or other suitable biometric data for use in performing portions of the method 100. In a specific example, the system 200 can include a biometric subsystem (e.g., a module of a remote server; a module of the medical improvement system 202, etc.) operable to collect a biometric dataset associated with a status of the user, where the biometric dataset is sampled for the first user at a biometric device (e.g., a user medical device), where the medical improvement subsystem 202 is operable to leverage the biometric dataset in performing portions of the method 100 (e.g., assigning the user to a user subgroup based on the biometric dataset and/or other suitable criteria such as one or more physical activity characteristics of the user; etc.). In another example, the system 200 can include an optical subsystem operable to collect an optical dataset associated with a foodstuff consumed by the user, where the optical dataset is sampled at an optical sensor (e.g., of a mobile device, such as a user smartphone) associated with the user, where the medical improvement subsystem 202 is operable to: facilitate processing of the optical dataset (e.g., transmission to a facilitator; automatic computational processing) to identify a foodstuff type (e.g., through computer vision techniques) associated with the foodstuff; and promote a therapeutic intervention to the first user based on the foodstuff. However, treatment systems can possess any suitable functionality.

Treatment systems can include any one or more of: motion sensor subsystems 203 (e.g., pedometers), weight sensor subsystems 201 (e.g., weight scales), blood sugar monitors, blood pressure monitors, devices associated with EEG, EOG, EMG, ECG, thermometers, heart rate monitors, ambient environment devices (e.g., such as sensing and control systems for temperature, light, air quality and/or composition, etc.), medication devices (e.g., such as automatic medication dispensers; personal assistant devices; etc.), user devices (e.g., through which application-based therapeutic interventions, such as curriculum components, can be promoted, etc.), facilitator devices, and/or any other suitable devices (e.g., biometric, medical and/or diagnostic devices, such as those configured to monitor and/or determine a wide variety of biometrics/biomarkers of an individual, etc.). In examples, treatment systems and/or other suitable system components can be used to receive or calculate biometric data about the participant. The biometric data may include, for example, blood sugar values, heart beat values, blood pressure, temperature, weight, body mass index values, body fat percentage, hydration, and/or other biometric data.

Treatment systems preferably promote therapeutic interventions for improving one or more user statuses. User statuses can include any one or more of: symptoms, causes, diseases, disorders, and/or any other suitable aspects associated with user conditions. In examples, user status can include health conditions such as obesity, pre-diabetes, heart disease, and/or other suitable health conditions. However, treatment systems and/or other portions of a medical improvement system 202 can be configured in any suitable manner.

### 3.3 System—Motion Sensor Subsystem.

The system 200 can additionally or alternatively include one or more motion sensor subsystems 203, which function to collect and/or transmit motion datasets for a set of users. A motion sensor subsystem 203 preferably includes one or more inertial sensors (and/or other suitable activity-related sensors), one or more communication modules (e.g. analogous to communication modules of the weight sensor subsystem 201; a wireless communication module operable to transmit motion datasets; etc.), and/or any other suitable components. Motion sensor subsystems 203 are preferably associated with a set of users (e.g., a motion sensor subsystem 203 assigned to a user; a motion sensor subsystem identifier stored in association with a user account, user subgroup, and/or other suitable component, etc.), and/or coupleable to a set of users (e.g., physically coupleable to a body region, etc.). However, motion sensor subsystems 203 can be associated with any suitable components in any suitable manner.

A motion sensor subsystem 203 preferably includes one or more inertial sensors, which function to sample motion datasets describing physical orientations associated with the user (e.g., physical orientations of a motion sensor subsystem 203 coupled to the user, where the physical orientation data can be processed to determine level of physical activity, a footstep parameter such as number of footsteps in a time period, etc.). In an example, the system 200 can include a set of motion sensor subsystems 203 including a first and a second inertial sensor, each mountable to a different user and operable to sample different motion datasets, which can be leveraged (e.g., by the medical improvement subsystem 202) to determine physical activity metrics and/or promote therapeutic interventions. Additionally or alternatively, the motion datasets can describe any suitable motion-related parameter. The motion datasets are preferably associated with one or more physical activity features of a user (e.g., associated with a user weight, diet, physical activity regiment, other suitable criteria upon which a user group can be determined; etc.). However, motion datasets can be associated with any suitable components.

The motion sensor subsystem 203 can include one or more: pedometers, data collection modules (e.g., as a component of the medical improvement system 202) operable to collect motion datasets sampled at remote inertial sensors (e.g., of user smartphones), and/or other suitable components. For example, the motion dataset can be sampled at an inertial sensor of a mobile device associated with the user, the mobile device including a microprocessor, a display, and a wireless communication transceiver, and where the medical improvement subsystem 202 (e.g., a motion sensor subsystem 203 of the medical improvement system 202) is operable to: wirelessly receive the motion dataset from the wireless communication transceiver of the mobile device; and present a visual representation of a physical activity metric (e.g., derived from the motion dataset) at the display of the mobile device. However, the motion sensor subsystem 203 can be configured in any suitable manner.

### 3.4 User Interface.

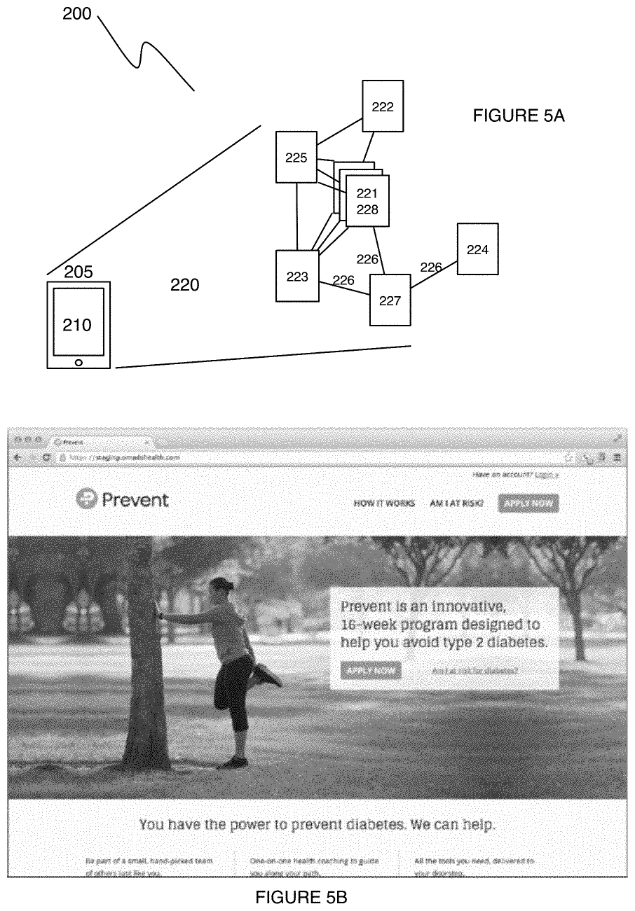

As shown in FIG. 5A, the system 200 can additionally or alternatively include a user interface 200 for supporting a health regimen, which can additionally or alternatively include a networked computing device 205 with a display 210, and an application 220 including a plurality of profile pages 221, each profile page corresponding to a respective participant in a first group participating in a health regimen, a progress page 222 accessible by a participant and configured to display health regimen progress of the participant, a first group page 223 corresponding to the first group and a second group page 224 corresponding to a second group, a curriculum page 225 configured to provide a health regimen curriculum to at least the participant, a message client 226 configured to provide communication between the participant and a second entity, and at least two modes, including a facilitator mode 227 and a participant mode 228, and/or any other suitable components. The user interface 200 functions to render an interactive environment by which participants in a health regimen may receive peer-based support and facilitator-based support, as well as guidance (in the form of a health regimen curriculum) and/or personalized information regarding health regimen progress. As shown in FIG. 1, the user interface is preferably coupled to a system for supporting a health regimen.

The application 220 functions to provide an interface by which a participant and/or a facilitator may receive information regarding health regimen progress of a participant and/or a group of participants, and may interact with another participant in order to provide a source of motivation in support of a health regimen. In a first variation, the application 220 is centrally hosted by one or more servers, and interacts with a plurality of networked computing devices 205 with displays 210, each networked computing device 205 corresponding to a participant. In a second variation, the application 220 is hosted by a distributed system, where at least one networked computing device 205 with a display 210 functions as a participant terminal, as a local server, or as both. The application may be a web application accessible through a web browser on a networked computing device 205, or may alternatively be a native application on the networked computing device 205. The application 220 preferably includes a plurality of profile pages 221, each profile page corresponding to a respective participant in a first group participating in a health regimen, a progress page 222 accessible by a participant and configured to display health regimen progress of the participant, a first group page 223 corresponding to the first group and a second group page 224 corresponding to a second group, a curriculum page 225 configured to provide a health regimen curriculum to at least the participant, a message client 226 configured to provide communication between the participant and a second entity, and at least two modes, including a facilitator mode 227 and a participant mode 228.

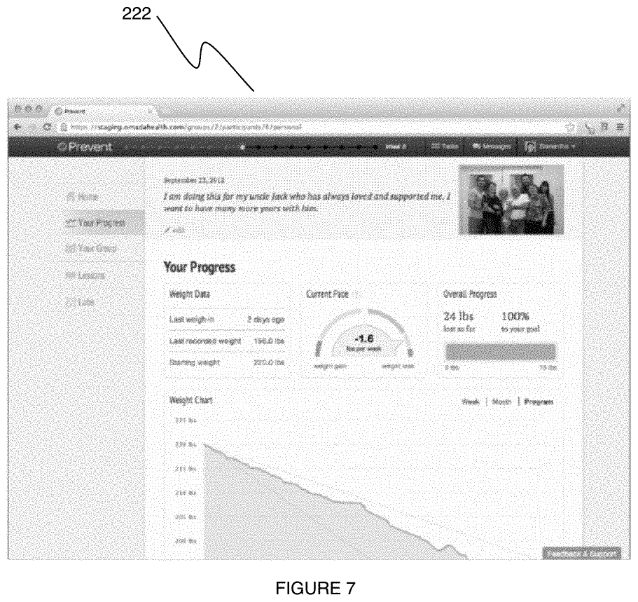

As shown in FIG. 7, the application 220 also includes a progress page 222 accessible by a participant and configured to display health regimen progress of the participant. The progress page 222 functions to display participant progress in the form of visuals and/or analyzed metrics as a source of motivation for a participant following a health regimen. The progress page 222 is preferably configured to display details and analyses of progress achieved by a given participant in the health regimen such as a trend in a body metric measurement of the participant, a trend in a body metric measurement of a participant relative to that of a matched group, and/or a target goal in the health regimen for the participant. The progress page 222 may be further configured to display overall progress achieved by a participant relative to certain earlier points and/or a starting point, a rate of progress (e.g. body metric change versus time), overall progress achieved by a participant relative to a goal, and/or other personalized biometric data (e.g. current weight, height, age, body mass index). Preferably, the progress page 222 is distinct from a profile page for a participant; however, alternatively, the progress page 222 and profile page for a participant are non-distinct pages. In an example, using metrics determined from an exercise tracking biometric device, such as a watch that records run time and distance, the user interface can present progress (e.g., physical activity metrics) indicating that one participant is underperforming relative to the established goal and/or relative to other users in the user subgroup. In a variation, the user interface can display progress in relation to sub-programs of a group program. Sub-programs can include one or more of: tasks (e.g., associated with diet, physical exercises, physical activity features, etc.), games, goals, and/or other suitable user actions. For example, a sub-program may include a task of “walking for one hour for each day in the week”, “eliminate sugary drinks and processed foods for the week”, and/or “sleep eight hours per night during the week”. In an example of displaying progress in relation to sub-programs, the medical improvement system 202 can be operable to determine a personal completion percentage for each human of the human subgroup based on a set of physical activity metrics (e.g., whether the physical activity metrics meet the weight loss goals and/or motion dataset-related goals associated with the sub-programs), where the personal completion percentage is associated with the set of sub-programs; and determine an aggregate completion percentage for the human subgroup based on the set of physical activity metrics (e.g., whether the aggregate weight loss of the user subgroup meets the aggregate weight loss goals, etc.); and where the user interface and/or other suitable component can be operable to present the personal completion percentage and the aggregate completion percentage. Additionally or alternatively, determining and/or presenting progress in relation to sub-programs and/or other suitable aspects of user health regimens can be performed in any manner analogous to U.S. application Ser. No. 14/190,017 filed 25 Feb. 2014 and U.S. application Ser. No. 14/245,961 filed 4 Apr. 2014, each of which are herein incorporated by this reference.

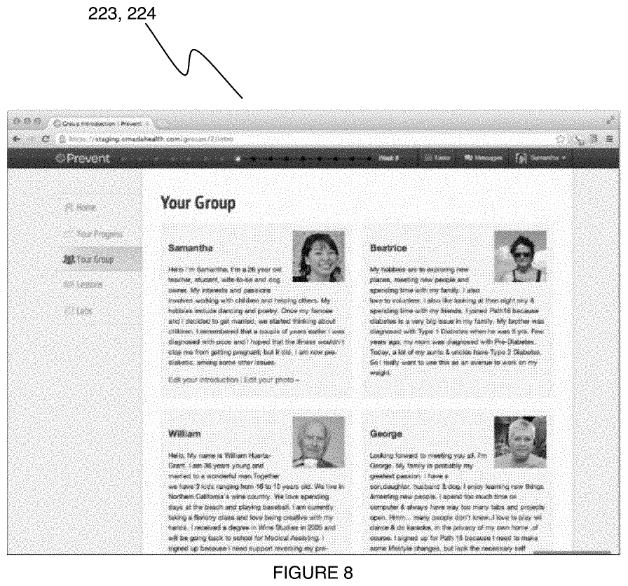

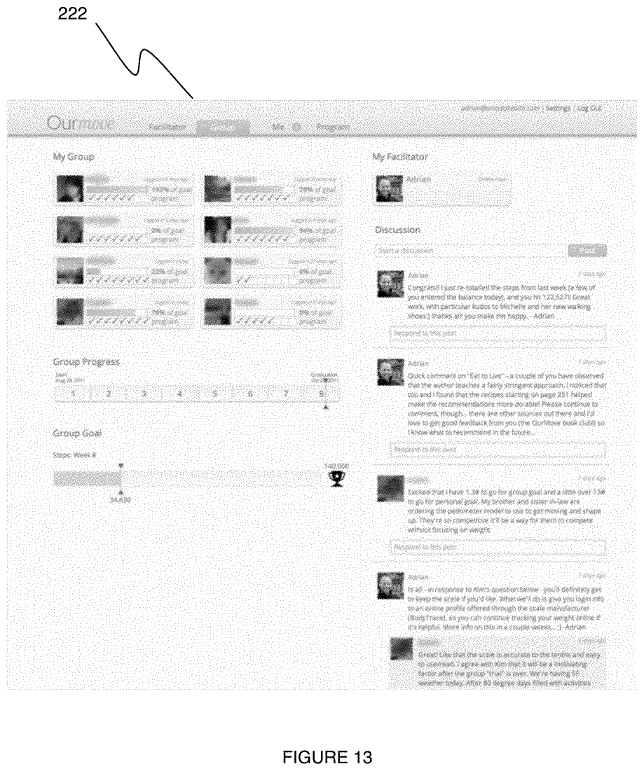

The application 220 can additionally or alternatively include a first group page 223 and a second group page 224 that each function to provide a centralized hub for interactions between participants of a group participating in a health regimen. As shown in FIGS. 8 and 13, a group page 223, 224 preferably displays a list and/or thumbnail summaries of the participants in a group participating in a health regimen, summary information about the progress of the group in the health regimen (e.g. trends and metrics determined from body metric measurement data), and any feedback addressed to the overall group from a facilitator and/or other participants. A group page 223, 224 preferably also includes links to profile pages of all participants of the group, and may further include information regarding the health regimen being followed by participants in the group. In alternative embodiments, a group page 223, 224 may only display a list and/or thumbnail summaries of the participants in a group participating in a health regimen, and links to profile pages corresponding to each member in the group participating in a health regimen, as shown in the example of FIG. 8.

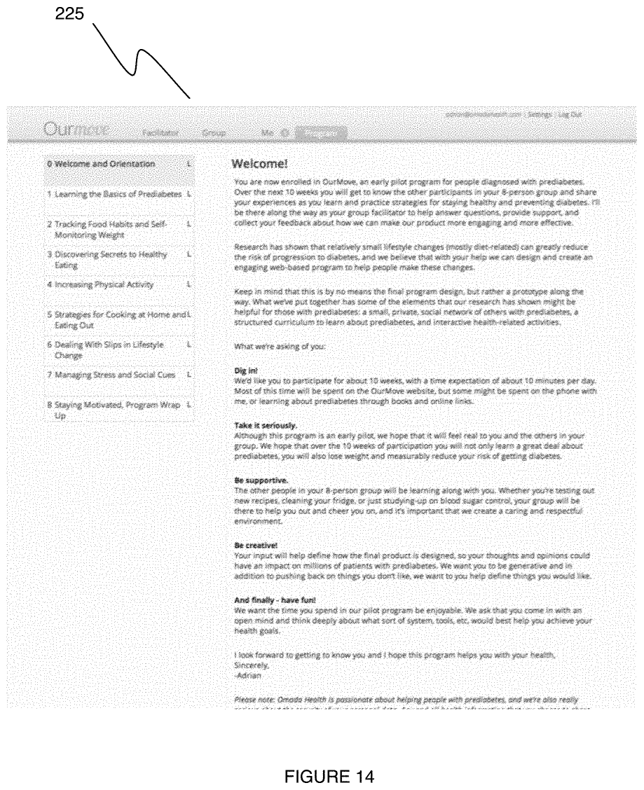

The application 220 can additionally or alternatively include a curriculum page 225 that functions to provide a health regimen curriculum intended to be followed by a participant. The curriculum page 225 preferably outlines steps or other features of a health regimen program. In the preferred embodiment, the curriculum page outlines steps based on the Diabetes Prevention Program (a research study funded by the National Institute of Diabetes and Digestive and Kidney Diseases), but in alternative embodiments, the curriculum page outlines steps or teaches lessons from other alternative health regimens. In an example, as shown in FIG. 14, the curriculum page 225 may include a welcome introduction to the program, tips, guidelines, and/or instructions corresponding to the health regimen program. In another example, as shown in FIG. 11, the curriculum page 225 may alternatively display health regimen tips in the form of a lesson plan, including modules, milestones, and/or assignments. Preferably, the curriculum page is configured to display the same curriculum for all participants in a group participating in a health regimen; however, alternatively, the curriculum page may be configured to display a curriculum that is customized to a given participant (e.g. based on participant performance). Preferably, the curriculum page 225 is accessible from a profile page 221, a progress page 222, and a group page 223, 224, but alternatively, the curriculum page 225 is accessible from a subset of a profile page 221, a progress page 222, and a group page 223, 224.

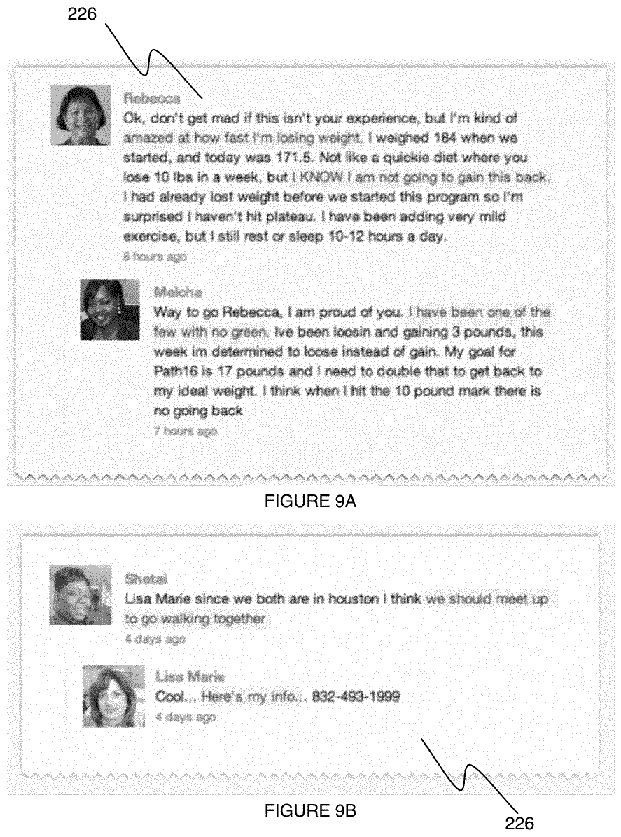

The application 220 can additionally or alternatively include a message client 226 that functions to enable communication between a participant and another entity, facilitated by the user interface. The message client preferably communicates with a server of a message service provider, server of a mailbox service that is a proxy for the message service provider, or any suitable messaging service. The message client preferably enables sending and receiving of messages, and may incorporate messages into a rendered interface. As shown in FIGS. 9A and 9B, the message client 226 may enable communication between a first participant and a second participant. In the example shown in FIG. 9A, a second participant may provide verbal motivational support to a first participant by describing a personal experience while following the health regimen. In the example shown in FIG. 9B, a first participant may connect with a second participant and set up a meeting to perform a task associated with a health regimen curriculum together. Additionally, the message client 226 may enable communication between a participant and a facilitator. In the example shown in FIG. 11, the facilitator may provide advice and motivational support to a participant through the message client 226, in a manner that is only accessible by the participant and the facilitator (i.e. no other participants have access to a communication between the participant and the facilitator). Preferably, either a participant or a facilitator may initiate a participant-facilitator communication by using the message client 226; however, alternatively, only the facilitator may initiate a participant-facilitator communication using the message client 226. The message client preferably also enables communication between more than two entities (e.g. a participant may communicate with at least two other participants, or at least one other participant and a facilitator). In variations, the coach or third party may utilize the system to provide a modification to a sub-program (and/or associated aspects, such as user goals, therapeutic interventions, etc.) when a participant is underachieving in the sub-program or a task associated with the sub-program.

The user interface preferably includes at least two modes, including a facilitator mode 227 that is activated by a facilitator, and a participant mode 228 that is activated by a participant. The facilitator mode 227 and the participant mode 228 function to provide a facilitator view of the user interface and a participant view of the user interface that is preferably generally more restricted than the facilitator view (except, for example, a particular participant may have an unrestricted view of his or her own profile page), respectively. The facilitator and/or participant modes 227, 228 enable levels of privacy and/or access to respective profile pages of participants. In an example, the user interface can be operable to improve display of the set of physical activity metrics, where the user interface can be operable between: a facilitator mode accessible by a facilitator at a facilitator device and restricted from the human subgroup, where the facilitator mode grants access to a first and a second display, where the first display includes a first subset of physical activity metrics from the set of physical activity metrics, and where the second display includes a second subset of physical activity metrics from the set of physical activity metrics; and a participant mode accessible by the human subgroup at corresponding user devices, where the participant mode grants access to the second display. In a specific example, the first subset of physical activity metrics includes current weights for each human of the human subgroup, and the second subset of physical activity metrics includes a weight loss percentage over time for each human of the human subgroup. In another example, in the facilitator mode 227 a facilitator of a group may have permission to view a physical activity metric both in percentage change and in absolute numbers, while in a participant mode 228 other participants of the group may be restricted to view only the physical activity metric in percentage change. In a second example, in the facilitator mode 227 a facilitator of a group may have access to all personal and/or biographic information corresponding to each participant in the group he or she facilitates, whereas in participant mode 228 a participant may only have access to his or her own personal and/or biographic information. Such restrictions are preferably set by the participant in a settings portal, as will be understood by one ordinarily skilled in the art. However, the user interface preferably enables each participant to set any suitable privacy and access settings to his profile page or other personal information.

In one embodiment, the facilitator mode 227 may further enable a facilitator to facilitate more than one group (e.g. the first and second group). The facilitator mode may thus include an additional facilitator page that enables the facilitator, using the message client 226, to communicate with all groups that the facilitator facilitates. The facilitator mode may enable the facilitator to communicate individually with members of the groups he/she facilitates, or to communicate with an entire group or portion of a group he/she facilitates. In a variation, the facilitator mode 227 may further enable a facilitator to have unrestricted viewing access to all profile pages and group pages corresponding to groups he/she facilitates, but may restrict the facilitator from modifying information displayed on the profile and group pages. In another variation, the facilitator mode 227 may enable a facilitator to have unrestricted viewing access to and the ability to modify all profile pages and group pages corresponding to groups he/she facilitates.

In other embodiments of the user interface 200, the first and second group pages 223, 224 may be further configured to provide a competition between the first group and the second group, in achieving a health regimen goal. In a first variation, a participant of the first group may compete with a portion of the participants of the second group, by accessing at least one of the first and second group pages 223, 224. In a second variation, the entire first group may compete with the entire second group, using at least one of the first and second group pages. Other embodiments of the user interface may incorporate additional pages, such as a home page, as shown in FIG. 5B, and/or functionality in the facilitator and participant modes 227, 228 to further support the health regimen. However, the user interface and/or associated components can be configured in any suitable manner.

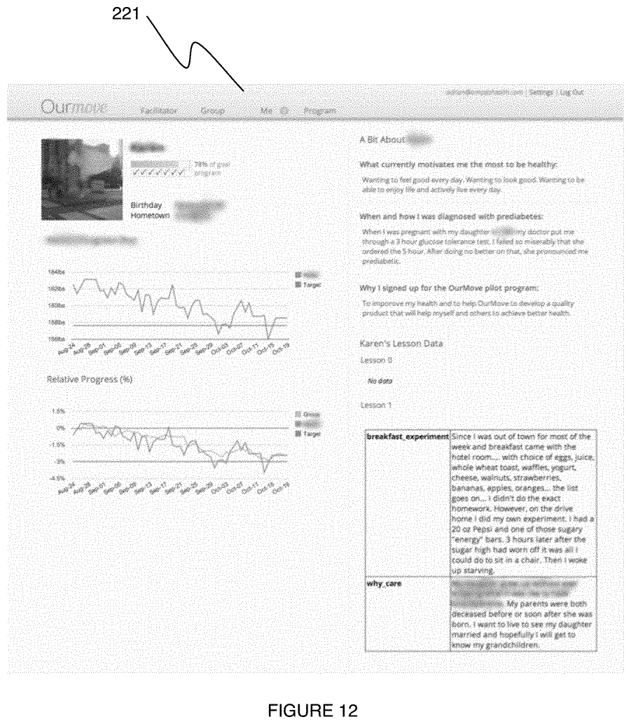

The system 200 can additionally or alternatively include components (e.g., as shown in FIGS. 12 and 17-20) described in U.S. application Ser. No. 14/180,205 filed 13 Feb. 2014, U.S. application Ser. No. 14/190,017 filed 25 Feb. 2014, and U.S. application Ser. No. 14/245,961 filed 4 Apr. 2014, each of which are herein incorporated in their entirety by this reference, and/or the system 200 can include any suitable components configured in any suitable manner.

### 4. Method.

As shown in FIG. 1, in embodiments, the method 100 for supporting a health regimen can additionally or alternatively, include: grouping a plurality of participants into a matched group S110; providing, to each participant of the matched group, a body metric measurement device configured to communicate remotely with a network S120; receiving a set of body metric measurement data over the network from a participant and a portion of the participants of the matched group S130; storing the set of body metric measurement data S140 on a server; determining a physical activity metric of the participant S150; determining a physical activity metric of the portion of the matched group S152; and/or providing feedback to the participant based on the physical activity metric of the participant relative to the physical activity metric of the portion of the matched group S160.

### 4.1 Method—Grouping Participants

Grouping a plurality of participants into a matched group S110 functions to establish a community among participants. The participants within a matched group preferably share at least one common goal related to a body metric measurement, such as losing weight, maintaining weight, gaining weight, or reducing body fat percentage, and/or a common goal related to a health condition, such as preventing development of prediabetes to diabetes. Alternatively the participants within a matched group are grouped based on another characteristic. In a preferred embodiment, a matched group includes approximately 8-16 participants, although the matched group may include any suitable number. Grouping a plurality of participants may include one or more variations that cluster participants in similar or the same groups based on various shared characteristics.

In a first variation of Block S110, grouping a plurality of participants into a matched group S110 includes grouping participants based on a characteristic of a common goal. In a first example of the first variation, the participants within a matched group may share the goal of losing or gaining a certain percentage (e.g. 5%) of an individual respective starting weight or a certain number of pounds. In a second example of the first variation, the participants within a matched group may share the goal of maintaining current starting weight or to attain a particular goal weight. In other examples of the first variation, the participants within a matched group may share the goal of losing, gaining, maintaining, or attaining a particular level or amount of BMI, body fat percentage, or other body metric measurement.

In a second variation of Block S110, grouping a plurality of participants into a matched group S110 includes grouping participants based on medical history. In a first example of the second variation, participants within a matched group may be diagnosed with a particular condition at approximately the same time (e.g. diagnosed with pre-diabetes within two months of one another, or another suitable threshold). In a second example of the second variation, participants within a matched group may have similar initial body weights, similar initial degree (class or stage) of congestive heart failure or other diagnosis of a cardiovascular disease. In a third example of the second variation, participants within a matched group may be diagnosed with a similar degree of obesity, and in a fourth example of the second variation, participants within a matched group may be diagnosed with a similar stage of osteoarthritis or other joint disease that affects mobility. Other aspects of medical history may be considered in matching participants, such as diagnosis of depression or obsessive-compulsive disorder.

In a third variation of Block S110, grouping a plurality of participants into a matched group S110 includes grouping participants based on shared personality traits, or similar positions within a personality spectrum. In an example of the third variation, participants within a matched group may have received similar results of a personality test or other assessment. Shared personality traits may include, for instance, optimism, extroversion, openness, agreeableness, or neuroticism. Grouping participants into a matched group may include administering to the participants a standard personality test (e.g. Myers-Brigg personality test, Big Five personality test) or a customized personality test, and clustering participants into matched groups based on the results of the standard or customized personality test.

In a fourth variation of Block S110, grouping a plurality of participants into a matched group S110 includes grouping participants based on a shared lifestyle characteristic or common interests. In an example of the fourth variation, participants within a matched group may have similar dietary restrictions or preferences (e.g., vegetarianism, veganism, nut-free, gluten-free), marriage status (e.g., married, divorced, widowed, single), children status (e.g. existence, age, gender, number of children), pet status (e.g. existence, age, species, number of pets), religious identification, or other suitable lifestyle characteristic. In another example of the fourth variation, the participants within a matched group may have similar hobbies or other interests (e.g. sports, television shows, cooking).

In a fifth variation of Block S110, grouping participants into a matched group includes grouping participants based on personal information. In examples of the fifth variation, such personal information may include gender, ethnicity or nationality, age, current geographical area, other location characteristics, or occupational field. As another example of the fifth variation, personal information may include hometowns, schools attended, employers, or any suitable personal information.

In additional variations of Block S110, the step of grouping participants may incorporate any suitable combination of these variations and/or any suitable aspect of the participants. In some embodiments of the method, the participants may additionally and/or alternatively be grouped based on contrasting or complementary aspects, rather than all common traits. For example, participants within a matched group may include both optimists and pessimists, or extroverts and introverts. Furthermore, the step of grouping participants may include weighting one or more of the various characteristics more heavily than others in their importance in the grouping process. For example, grouping participants based on a characteristic of a common goal is preferably weighted more heavily than grouping participants based on personal information.

Grouping a plurality of participants into a matched group S110 may further include sorting the participants using a “tiered” or “staged” process that effectively places the various characteristics in a hierarchy of importance. For instance, in a first stage an initial group of participants may filtered into a second group of participants that exclusively share the goal of losing a particular percentage of their initial respective weights. In a second stage, the second group of participants may be further filtered into a third group of participants that are within a particular age range. In a third stage, the third group of participants may be further filtered into a fourth group of participants that are of the same gender. In this manner, the grouping process may include any suitable number of stages that successively reduce or sort a larger group of participants into smaller matched groups until one or more suitable matched groups are created. In another embodiment, grouping may additionally and/or alternatively include assigning each of the participants a classification or number based on the sorting characteristics and grouping the participants based on their respective classification or number. However, the sorting characteristics may be used to group participants into appropriate matched groups in any suitable manner.

### 4.2 Method—Providing a Body Metric Measurement Device.

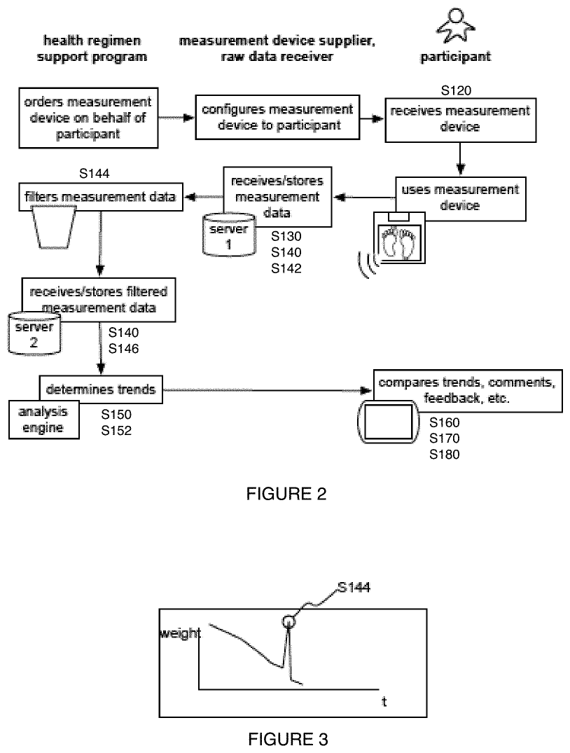

Providing, to each participant of the matched group, a body metric measurement device configured to communicate remotely with a network S120, that functions to facilitate measuring a body metric of the participant and to facilitate a manner in which the participants can submit or communicate their body metric measurements (also referred to more simply as “measurements”, “measurement data”, or data points) to a server. Preferably, the body metric measurement device is a weight scale that measures the body weight of a participant. For example, the body metric measurement device may be a BodyTrace™ eScale. In alternative embodiments, the body metric measurement device may be a body fat measuring device (e.g. skinfold caliper), a sphygmomanometer that measures blood pressure, a blood glucose monitor, or any suitable body metric measuring device. Furthermore, the method 100 may further include providing multiple body metric measurement devices (e.g., a weight scale that communicates weight of the participant and a pedometer that communicates number of steps walked by the participant) to each participant of the matched group. Preferably, the body metric measurement device requires no user setup (e.g. calibration and setup performed before the user receives the device, as shown in FIG. 2), but alternatively, minimal setup by the user may be required (e.g. input of identification information prior to device activation). In some embodiments, as shown in FIG. 2, the body metric measurement device may be electronically paired or assigned to a particular participant, such as by linking a product serial number with the name of the participant and storing the link information in a database. The body metric measurement device is preferably configured to communicate over a network such that body metric measurement data may be uploaded to a remote storage, such as through cellular networks (e.g., Global System for Mobile Communications) or over the internet (e.g., Wi-Fi). Preferably, identical models of a body metric measurement device are provided to all participants within a matched group, to maintain consistency and comparability of measurements between participants. Providing identical models of the body metric measurement device may further include calibrating all models provided to participants of a matched group, such that they perform consistently in relation to each other.

### 4.3 Method—Receiving Body Metric Measurement Data.

Receiving a set of body metric measurement data S130 over the network from the participant and a portion of the participants of the matched group functions to gather data from which to generate feedback in support of the health regimen. This step is preferably repeated over time such that a time series of body metric measurement data may be received in regular intervals (e.g., hourly, daily, weekly, biweekly) or irregular intervals from the participant and at least one other participant of the matched group. The set of body metric measurement data may further include multiple time series of body metric measurement data, the multiple time series of body metric measurement data including a time series from the participant, and a time series from each participant of the portion of the matched group. Measurements from the participant and from each participant of the portion of the matched group may be received at the same time or at different times; preferably, measurements from the participant and from each participant in the portion of the matched group are received at the same frequency and/or simultaneously. Alternatively, measurements from the participant and from each participant in the portion of the matched group are received at different frequencies and/or different instances. As described above, the multiple time series are preferably received over a network such as a Global System for Mobile Communication or Wi-Fi. Each body metric measurement in the set of body metric measurement data is preferably labeled with identifying information, such as date, time, and/or location of measurement, personal information identifying the participant being measured, and/or a serial number or other identifier of the body metric measurement device. A time series of measurements is preferably received with push technology, such that the measurement device of a participant initiates transmission of body metric measurement data. However, the time series of measurements may additionally and/or alternatively be received with pull technology, such that the receiver initiates transmission of the body metric measurement (e.g. through polling or manual initiation on the receiver side). A time series of body metric measurements may be received as individual measurements, or as packets or bundles of multiple measurements.

### 4.4 Method—Storing Body Metric Measurement Data.

Storing the set of body metric measurement data S140 on a server or other database functions to create and maintain a record of received measurement data from the participant and one or more of the participants of the matched group. Storing the set of body metric measurement data S140 enables the set of body metric measurements, including at least one time series of data, to be shared.

Storing the set of body metric measurement data S140 on a server preferably includes filtering the received set of body metric measurement data S144, which functions to remove any suspicious measurements from the received measurement data. In particular, filtering preferably includes identifying erroneous measurements. Example erroneous measurements include measurements that are unlikely to come from a participant (e.g. measurements resulting from outsider interference), erroneous measurements due to device malfunction, erroneous measurements due to participant error, and other non-representative measurements. In one embodiment, the method 100 may further include detecting if an outsider has used the device (e.g. through identity verification), so as to produce an erroneous measurement. As shown in FIG. 3, identifying erroneous measurements may include analyzing for unrealistic measurement gains or losses (outliers) compared to previously determined physical activity metrics. In a first example of filtering the received set of body metric measurement data S144, a single body metric measurement may be identified/flagged if the measurement indicates a significant weight gain of 10 pounds over one day relative to the average weight of the previous 5 days. In a second example of filtering the received set of body metric measurement data S144, any body metric measurement in the received set of body metric measurement data may be identified/flagged if the measurement deviates from an adjacent measurement by a specified amount. In a third example of filtering the received set of body metric measurement data, a line may be fitted to the set of body metric measurement data, and any measurement that has a residual (relative to the line) with an absolute value greater than a specified amount may be identified/flagged. However, any suitable analysis for filtering the received measurements may be performed. The identified/flagged measurements may be automatically removed from the data set or marked for manual review and removal from the data set. In some variations, the degree to which a flagged measurement is suspicious may affect whether the flagged measurement is automatically removed or marked for review (e.g., flagged measurements that deviate from the trend by a certain threshold amount are automatically removed from the data set).

In a variation, Block S140 can include associating body metric measurement data with any suitable identifiers, and/or otherwise associating data. For example, the method 100 can include: associating a user account with a user subgroup identifier, where the user account identifies the first human and is operable to improve personalization of content delivered to the first human, and where the user subgroup identifier identifies a user subgroup that the user is assigned to; and associates a body metric measurement dataset with the user account and the human subgroup identifier. However, Block S140 can be performed in any suitable manner.

### 4.5 Method—Determining a Physical Activity Metric.

Determining a physical activity metric of the participant S150 functions to determine one or more metrics indicative of the progress and/or status of the participant in the health regimen (e.g., as a function of time), in relation to a user status of the user, and/or in relation to any suitable aspect associated with the user. Physical activity metrics can include one or more of: weight-related metrics (e.g., weight, average weight over time, percentage weight loss in relation to a weight loss goal, BMI, weight metrics in relation to a user subgroup, etc.), motion-related metrics (e.g., in forms analogous to weight-related metrics), body metric measurement trends (e.g., generated from a series of a body metric measurement data points collected over time; across a plurality of users in a user subgroup; etc.), other physical activity-related metrics derived from body metric measurement data, and/or any other suitable metrics.

Determining physical activity metrics S150 is preferably based on one or more body metric measurement datasets (e.g., weight datasets, motion datasets, etc.), but can additionally or alternatively be based on one or more of: user subgroups (e.g., body metric measurement datasets for other users in the user subgroups; aggregating total weight loss over a period of time across the users in a user subgroup; otherwise combining datasets across users in a user subgroup to indicate progress for an individual user or set of users associated with a user subgroup; etc.), biomarkers, therapeutic interventions (e.g., determining physical activity metrics indicating effectiveness of a promoted therapeutic intervention, etc.), user demographics, user responses to surveys, and/or any other suitable data. In a variation, determining a physical activity metric can include: obtaining, applying, and/or otherwise manipulating a computer-implemented rule operable to improve processing by the medical improvement system (e.g., of body metric measurement datasets). Computer-implemented rules can include feature engineering rules, user preference rules (e.g., privacy rules associated with the types of body metric measurement datasets can be used, shared, and/or otherwise processed, etc.), user subgroup determination rules (e.g., parameters for matching users to user subgroups), facilitator matching rules (e.g., for assigning a facilitator to a user subgroup), therapeutic intervention rules (e.g., for promoting therapeutic interventions), and/or any other suitable computer-implemented rules enabling performance of the method 100. In a specific example, the method 100 can include generating a physical activity feature (e.g., an amount of weight loss and degree of physical activity over the past week) from evaluating the first weight dataset and the motion dataset against the feature engineering rule; and generating the physical activity metric (e.g., a cardiovascular health metric, etc.) based on the physical activity feature. However, computer-implemented rules can be used in facilitating any suitable portion of the method 100 (e.g., extracting features for determining therapeutic interventions, etc.), and can be configured in any suitable manner.

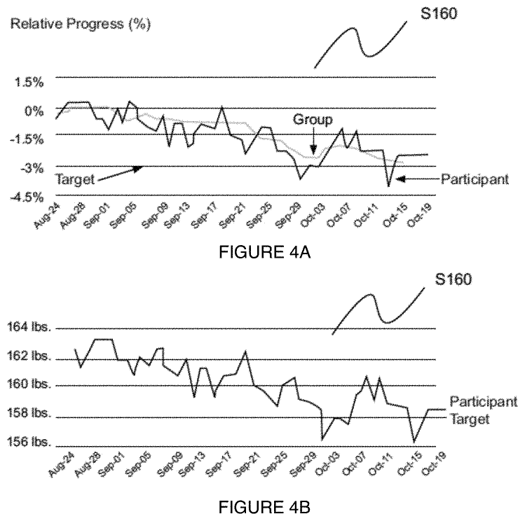

Regarding Block S150, a physical activity metric is preferably subsequently stored on at least one of the servers for future use (e.g., filtering future received measurements), but alternatively, an additional server may be used to store a physical activity metric. Determining a physical activity metric of the participant S150 may include one or more of several variations: In a first variation, as shown in FIG. 4A, measurements used to determine the physical activity metric of the participant are analyzed and output as percentages relative to an initial baseline measurement. In an example of the first variation, following an initial baseline weight measurement of 200 pounds, a subsequent measurement of 195 pounds (loss of five pounds) is calculated as a data point of 2.5% loss relative to the initial baseline weight in a weight trend. Additional subsequent measurements based on the set of body metric measurement data are analyzed relative to the initial baseline weight measurement. In a second variation, as shown in FIG. 4B, measurements used to determine the physical activity metric of the participant are analyzed and output as absolute differences relative to an initial baseline measurement, similar to the first variation; however, in the second variation, measurements are expressed as absolute numbers rather than percentages. In a third variation, measurements used to determine the physical activity metric of the participant are determined as percentages relative to a previous measurement, or an averaged (e.g., mean or median) value of a certain number of previous measurements in a time series of body metric measurement data. In a fourth variation, measurements used to determine the physical activity metric of the participant are determined as absolute differences relative to one or more previous measurements, similar to the third variation; however, in the fourth variation, data points are expressed as absolute numbers rather than percentages. In a fifth variation, a line may be fitted to body metric measurements for the participant, and a rate of progress (e.g. weight loss per unit time) may be used to represent the physical activity metric of a participant.

Determining a physical activity metric S150 of a portion of the matched group S152 functions to assess the progress or status of the matched group in the health regimen. Determining a physical activity metric of a portion of the matched group preferably includes determining a physical activity metric based on a set of body metric measurement data representing all participants in the matched group or alternatively, less than all participants in the matched group. The physical activity metric for the portion of the matched group may be calculated in a manner similar to calculating the physical activity metric of a single participant using any suitable variation as described above, except that each measurement/data point for the portion of the matched group may be an averaged (e.g., mean or median) measurement value of all of the participants within the matched group. In a first example using averaged measurement values, a time series of body metric measurement data may be collected from each participant of the portion of the matched group, and measurements taken at similar time points (e.g. within a 24-hour period of time in a 16 week time period) may be averaged across all participants of the portion of the matched group for use in determining the physical activity metric of the matched group. In a second example using averaged measurement values, the physical activity metric of the matched group may include a different number of measurements than the number of measurements used to determine a physical activity metric in a body metric measurement of the participant S150, as measurements from the participants in the portion of the matched group may not be available for identical periods of time (e.g. measurements are received once per day from one participant and once every two days from another participant). In the second example, the physical activity metric of the matched group may include a set of measurements, each representing an average group value over a two-week period, while the physical activity metric of the participant may include a set of measurements, each measurement representing a daily value. However, both the physical activity metric of the participant and the physical activity metric of a portion of the matched group may have any suitable resolution of measurement data points. In a third example averaged measurement values, each corresponding to different time points for the portion of the matched group, may be fitted to a line, such that a rate of progress of the portion of the matched group (e.g. weight loss per unit time) may be used to represent the physical activity metric of the portion of the matched group. Preferably, the participant is a part of the portion of the matched group, such that the body metric measurement data of the participant is factored into determining the physical activity metric in the body metric measurement data of the portion of the matched group; however, alternatively, the physical activity metric in the body metric measurement of the portion of the matched group may be determined from a subset of the set of body metric measurement data, where the subset excludes the body metric measurement data of the participant.

In variations, portions of the method 100 can be performed based on, in relation to, and/or in any suitable relationship to physical activity metrics satisfying threshold conditions (e.g., a weight loss rate falling below a threshold condition). In an example, the method 100 can include determining the therapeutic intervention in response to the physical activity metric falling below a threshold condition (e.g., where the therapeutic intervention includes at least one of a therapeutic drug, medical device operation, a diet, and a physical activity regimen, etc.). Additionally or alternatively, performing portions of the method 100 in relation to the values of the physical activity metrics can be performed in any manner analogous to that described in relation to U.S. application Ser. No. 14/245,961 filed 4 Apr. 2014, which is incorporated in its entirety by this reference, and/or performed in any suitable manner. However, determining physical activity metrics can be performed in any suitable manner.

### 4.6 Method—Providing Feedback.

Providing feedback to the participant S160 based on the physical activity metric functions to use the physical activity metric to support and motivate a participant during his or her health regimen. Preferably, the participant is a part of the matched group, such that the participant is motivated by fellow “team members” in the matched group to adhere to the health regimen. In a variation, the participant, as part of the matched group, “competes” against other matched groups as a source of support and motivation during his or her health regimen. Alternatively, the participant is not a part of the matched group, such that the participant “competes” against the matched group as a source of motivation during his or her health regimen. Preferably, feedback is provided through a user interface (described further below in more detail) communicatively coupled to at least one server that stores body metric measurements of the participants. The user interface is preferably an application accessed through a computing device, or alternatively, a website presented as a separate online social network site or online community. The user interface may alternatively be hosted by a third-party social network site. Providing feedback may include one or more of several steps as described below; however, the feedback may be provided in any suitable manner.

As shown in FIGS. 4A and 4B, providing feedback to the participant S160 preferably includes displaying the physical activity metric in the body metric measurements of the participant and/or displaying the physical activity metric in the body metric measurements of the matched group. One or both of these physical activity metrics may be displayed on a profile page of the participant in a user interface. The physical activity metrics are preferably displayed on charts as a function of time, with any suitable time divisions (e.g., daily, biweekly, weekly, monthly). The physical activity metrics may additionally and/or alternatively be displayed as tables, bar graphs, or in any other format. In an embodiment, the method 100 follows a designated health regimen program such as the Diabetes Prevention Program, and providing feedback to the participant S160 further includes displaying individual and/or group progress in the health regimen program and metrics of any activities associated with the health regimen, such as walking (e.g. determined using a connected pedometer). Simultaneously displaying physical activity metrics of a participant and of the matched group enables the participant to directly compare his or her progress and success in the health regimen with that of other participants, at least relative to the overall progress of the matched group. The overall progress of the matched group and individual progress of other participants in the matched group may be motivational to a particular participant, and are preferably relevant to a particular participant because of the nature in which the participants were sorted and grouped.

Providing feedback to the participant S160 preferably further includes enabling a facilitator associated with the matched group to access the physical activity metric of the participant and/or the physical activity metric of the portion of the matched group. Similarly, providing feedback to the participant S160 preferably further includes enabling one or more of the participants in the matched group to view a displayed physical activity metric of another participant and/or the physical activity metric of a portion of the matched group. However, providing feedback to the participant S160 may further include allowing the participant to designate privacy settings that limit the details available to other participants and/or the facilitator. For example, the participant may select settings such as to enable the facilitator and/or other participants to view a physical activity metric of his weight measurements represented in percentage of change, but to restrict the facilitator and/or other participants from viewing a physical activity metric of his/her weight measurements represented in absolute numbers.

Providing feedback to the participant S160 preferably includes promoting one or more therapeutic interventions, which functions to determine, provide, and/or otherwise facilitate therapeutic intervention provision to one or more users for improving user status. Promoting therapeutic interventions can include one or more of: generating control instructions (e.g., for operating one or more treatment systems, weight sensor subsystems, motion sensor subsystems, etc.); communicating with devices (e.g., transmitting control instructions, user interface components; receiving sensor data from treatment systems; etc.); controlling and/or operating system components; retrieving data (e.g., body metric measurement datasets for users of a user subgroup based on a user subgroup identifier, in order to generate an aggregate physical activity metric; etc.); and/or any other suitable operation. Types of therapeutic interventions can include any one or more of: physical activity-related notifications (e.g., including curriculum components, physical activity metrics, etc.); physical exercises, mental exercises; interactions with facilitators; medication interventions; mobile device and/or treatment system-related interventions (e.g., modifying device operation parameters; etc.); ambient environment interventions (e.g., modification of light parameters, air quality and/or composition parameters, temperature parameters, humidity parameters; etc.) and/or any other suitable types of interventions. In an example, promoting a therapeutic intervention can include activating an application executable on a mobile device associated with the user; and providing the therapeutic intervention through the application (e.g., in association with presenting the visual representation of the physical activity metric at the application, such as in parallel, in serial, etc.).

In relation to Block S160, promoting therapeutic interventions is preferably based on one or more physical activity metrics (e.g., recommending an increased frequency of outdoor walks based on a physical activity metric indicating a lower than average number of footsteps relative the user subgroup, etc.), but can additionally or alternatively be based one or more of: user demographic (e.g., therapeutic interventions correlated with positive outcomes for particular demographics, etc.), user subgroup (e.g., tailored to the shared physical activity characteristics of the user subgroup, tailored to involve communications and/or other suitable interactions, such as group exercise classes, between users of the user subgroup and/or facilitators for the user subgroup, etc.), therapeutic intervention effectiveness (e.g., adjusting therapeutic interventions, such as medication regimen aspects based on user response to administered medication), and/or any other suitable criteria (e.g., data used in determining physical activity metrics, etc.). In examples, the therapeutic intervention can include a personalized therapeutic intervention for the user (e.g., determined based on the physical activity metrics generated specifically for the user based on collected body metric measurement datasets for the user, etc.).

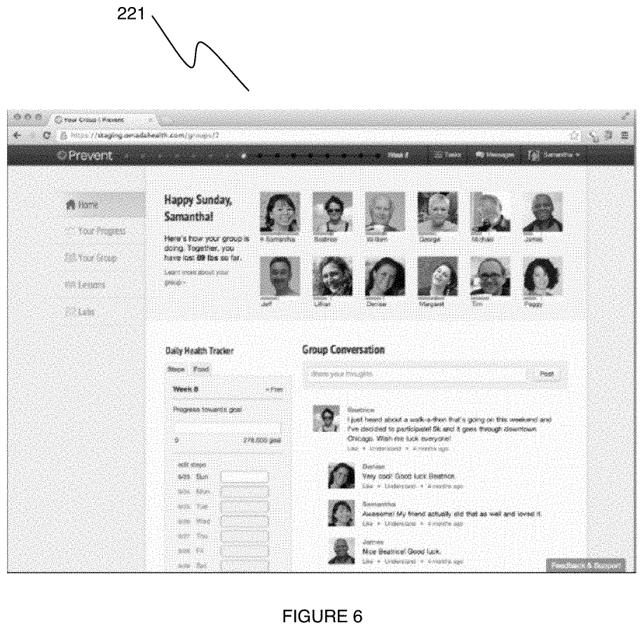

In a variation of Block S160, promoting a therapeutic intervention can include enabling a facilitator associated with the matched group to communicate with one or more of the users in the matched group. For example, promoting a therapeutic intervention can include: enabling a wireless communication link between a facilitator device and a user device, where the facilitator device is associated with a facilitator for the user subgroup, and where the user device is associated with the first user; and facilitating a video communication between the facilitator and the first user over the wireless communication link. As shown in FIG. 6, in another example, the facilitator may address general comments to the matched group on a group page of a user interface. The facilitator may additionally and/or alternatively provide targeted comments to a particular individual participant, such as by posting comments on the profile page of the participant, and/or by sending a personalized message accessible only by the individual participant and the facilitator. Similarly, providing feedback may further include enabling a participant in the matched group to provide comments to one or more of the other participants in the matched group, including general comments on the group page, targeted comments on the profile page of a particular targeted participant, and/or personalized messages accessible only by the participant and the targeted participant. Comments from the facilitator and fellow participants in the matched group serve to provide motivation and support throughout the health regimen. Such comments may include, for example, congratulatory remarks on a completed milestone, suggestions for modifications in activities (diet, exercise plan, etc.), general motivational remarks, sharing of personal stories to enhance personal connections within the matched group and/or facilitator, questions to generate discussions, invitations to perform a health regimen curriculum task socially, or any suitable comments. In some embodiments, providing feedback further includes enabling a facilitator and/or participants in the matched group to share photos or other media with another participant or the matched group in general. However, communications between users and/or facilitators can be in any suitable form (e.g., visual, audio, haptic, textual, virtual reality, etc.). Further, facilitating communications can be performed in any suitable manner.

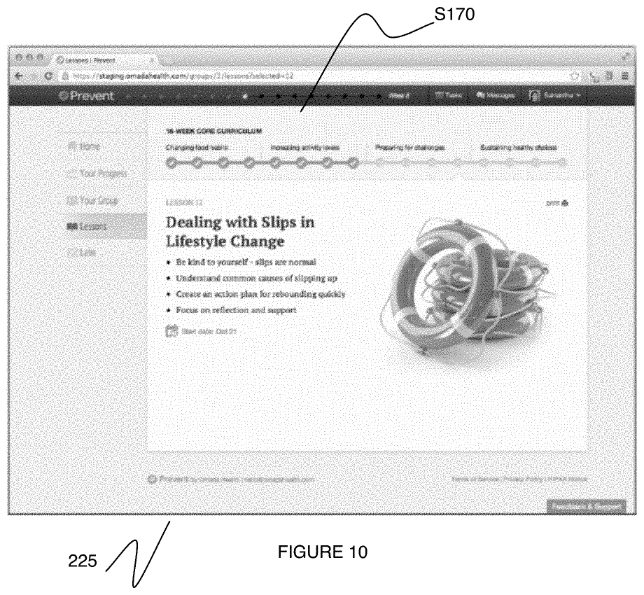

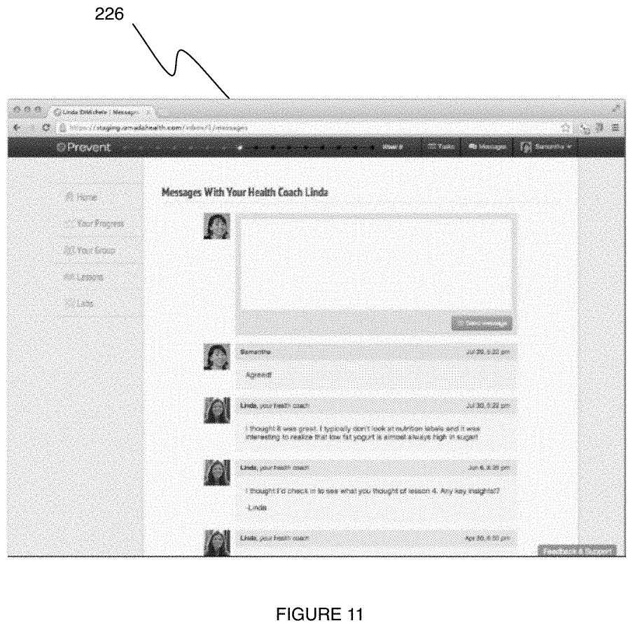

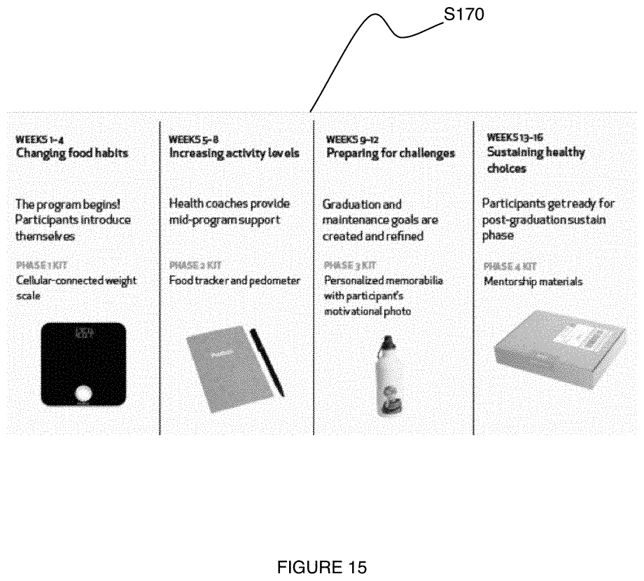

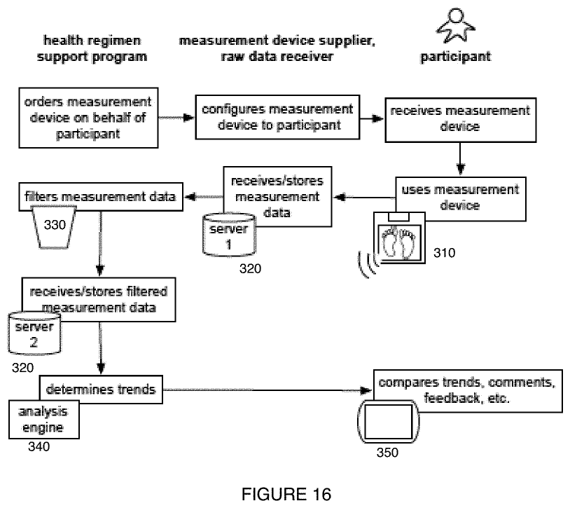

In a variation, promoting a therapeutic intervention can include providing a health regimen curriculum S170 (e.g., to each participant of the matched group, etc.), which functions to change a participant's eating and activity in order to achieve a goal. In a first example, the health regimen curriculum includes steps outlined in the Diabetes Prevention Program (a research study funded by the National Institute of Diabetes and Digestive and Kidney Diseases), and providing a health regimen curriculum includes presenting steps based on the Diabetes Prevention Program as lessons through a user interface. In the first example, as shown in FIGS. 10 and 15, the lessons may be organized into four phases, including: a first phase involving changing food habits, a second phase involving increasing activity levels, a third phase involving preparing for challenges, and a fourth phase involving sustaining healthy choices; furthermore, the participant may be encouraged to set goals and meet milestones, as well as complete assignments (e.g. journal entries, meal experiments) as part of the health regimen curriculum in the first example. The first example providing each of the four phases of lessons may be accompanied by providing a kit corresponding to each phase, where the first phase kit includes a body metric measurement device (e.g. a network-connected weight measurement device), the second phase kit includes a second measurement device and tool (e.g. a pedometer and a food tracking tool), the third phase kit includes motivational prizes (i.e. upon graduating from the curriculum), and the fourth phase kit includes materials to support the participant in sustaining healthy choices (i.e. post-graduation). In a second example, providing a health regimen curriculum S170 may include providing a diet modification and exercise routine regimen including daily meal plans and exercise tasks geared to treat a diagnosed condition, such as cardiovascular disease or diabetes. In a third example, providing a health regimen curriculum S170 may include providing a physical therapy regimen curriculum. In other examples, providing a health regimen curriculum S170 may include providing any appropriate health regimen curriculum for a given condition, that is preferably fixed, or alternatively, customizable by a participant, facilitator, or automatically to meet the participant's specific needs. The health regimen may be customizable by a facilitator or automatically, such that if the participant is not making progress at a rate comparable to that of a matched group, the health regimen may give the participant additional feedback and advice so that the participant is given an advantage or “handicap” relative to the matched group. The customized health regimen may be provided based on a performance metric of the participant, such as absolute change in body weight relative to an initial baseline measurement (after a period of time has elapsed from initiation of the regimen) or an unmet goal set by the participant and/or a facilitator.

In another variation, promoting a therapeutic intervention can include providing a physical motivational incentive to the participant S180, which functions to promote adherence to the health regimen curriculum. Providing a physical motivational incentive to the participant S180 may include providing health-related physical awards, such as coupons, nutritional supplements, and/or exercise equipment. In an example, providing a physical motivational incentive to the participant S180 may be performed after the participant has reached a health regimen goal/milestone, or if the participant experiences a quantifiable level of progress above a specified threshold. In an alternative example, providing a physical motivational incentive to the participant S180 may be performed if the participant is not making progress at a rate comparable to that of a matched group, such that the participant is given an advantage or “handicap” relative to the matched group to equalize chances of success relative to the matched group. The physical motivational incentive may be provided based on a performance metric of the participant, such as absolute change in body weight relative to an initial baseline measurement (after a period of time has elapsed from initiation of the regimen) or an unmet goal set by the participant and/or a facilitator. Additionally or alternatively, promoting a therapeutic intervention and/or other suitable aspects of providing user feedback can be analogous to U.S. application Ser. No. 14/245,961 filed 4 Apr. 2014, which is herein incorporated in its entirety by this reference.

In some alternative embodiments of the method 100, the method 100 may omit matched groups. For example, displaying feedback may include displaying the physical activity metric of a body metric measurement of a participant on the profile page of that participant, but not displaying a physical activity metric of the body metric measurement of any other participant or group of participants. By omitting matched groups, a facilitator may be assigned to work one-on-one with a participant, instead of in a group setting. However, the functionality of the system 200 can be distributed in any suitable manner amongst any suitable system components.

The system and method of the preferred embodiment and variations thereof can be embodied and/or implemented at least in part in the cloud or as a machine configured to receive a computer-readable medium storing computer-readable instructions. The instructions are preferably executed by computer-executable components preferably integrated with the system 100 and one or more portions of the processor and/or a controller. The computer-readable medium can be stored on any suitable computer-readable media such as RAMs, ROMs, flash memory, EEPROMs, optical devices (CD or DVD), hard drives, floppy drives, or any suitable device. The computer-executable component is preferably a general or application specific processor, but any suitable dedicated hardware or hardware/firmware combination device can alternatively or additionally execute the instructions.

The FIGURES illustrate the architecture, functionality and operation of possible implementations of methods according to preferred embodiments, example configurations, and variations thereof. In this regard, each block in a flowchart or block diagram may represent a module, segment, portion of code, or method step, which includes one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The method and system include every combination and permutation of the various system components and the various method processes, including any variations, embodiments, examples, and specific examples. As a person skilled in the art will recognize from the previous detailed description and from the figures and claims, modifications and changes can be made to the preferred embodiments of the invention without departing from the scope of this invention defined in the following claims.

We claim:

1. A method comprising:
determining a set of user baseline data for a user;
obtaining a motion data time series for the user, wherein the motion data time series comprises data acquired via a motion sensor;
obtaining a weight data time series for the user, wherein the weight data time series comprises data acquired via a weight sensor;
determining a set of user response data for the user;
generating a physical activity feature for the user based on: a set of feature engineering rules, the set of user baseline data, features extracted from the motion data time series, and features extracted from the weight data time series;
clustering the user into a group of users based on the physical activity feature;
calculating a group physical activity parameter based on a set of physical activity features generated for a plurality of users within the group;
automatically determining a set of optimized therapy instructions for the user based on the group physical activity parameter, the set of user baseline data, the user response data, the motion data time series, and the weight data time series; and
automatically controlling a user interface based on the set of optimized therapy instructions.

2. The method of claim 1, wherein the group physical activity parameter is not calculated based on the physical activity feature for the user, and wherein the set of optimized therapy instructions is further determined based on a comparison between the group physical activity parameter and a user physical activity parameter determined based on the physical activity feature for the user.

3. The method of claim 1, wherein a set of user baseline data for each user in the plurality of users within the group comprises a weight loss goal for said user, and wherein the group physical activity parameter is further calculated based on a percentage weight loss for each user relative to the weight loss goal for said user.

4. The method of claim 1, wherein the physical activity feature for the user is calculated based on a calculated trend relationship in the motion data time series and the weight data time series.

5. The method of claim 4, wherein the calculated trend relationship between the motion data time series and the weight data time series is evaluated on a one-week timescale.

6. The method of claim 1, wherein the set of optimized therapy instructions is calculated for at least one of a set of pathologies, wherein the set of pathologies comprise diabetes and obesity.

7. The method of claim 1, further comprising calculating a therapy efficacy parameter based on: a previous set of optimized therapy instructions for the user, the motion data time series, and the weight data time series, wherein the set of optimized therapy instructions is additionally determined based on the therapy efficacy parameter.

8. The method of claim 1, further comprising filtering the weight data time series, comprising:
calculating a weight trend in the weight data time series; and
establishing a flagging threshold model wherein a datapoint within the weight data timeseries is flagged when a calculated deviation from the weight trend is greater than a threshold value set by the flagging threshold model.

9. The method of claim 8, further comprising establishing a removal threshold model, wherein the datapoint is flagged for automatic removal when the calculated deviation is greater than a threshold value set by the removal threshold model.

10. The method of claim 1, further comprising supplying a plurality of users in the group with identical weight sensors, each uniquely and statically associated with a user.

11. A system comprising:
a motion sensor associated with a user;
a weight sensor associated with the user; and
a therapy instruction generation subsystem, configured to:
obtain a motion data time series, wherein the motion data time series comprises data acquired via the motion sensor;
obtain a weight data time series, wherein the weight data time series comprises data acquired via a weight sensor;
determine a set of user baseline data for the user;
determine a set of user response data for the user;
generate a physical activity feature for the user based on: a set of feature engineering rules, the set of user baseline data, features extracted from the motion data time series, and features extracted from the weight data time series;
cluster the user into a group of users based on the physical activity feature;
calculate a group physical activity parameter based on a set of physical activity features generated for a plurality of users within the group;
automatically determine a set of optimized therapy instructions for the user based on the group physical activity parameter, the set of user baseline data, the user response data, the motion data time series, and the weight data time series; and
automatically control a user device based on the set of optimized therapy instructions.

12. The system of claim 11, wherein the group physical activity parameter is not calculated based on the physical activity feature for the user, and wherein the set of optimized therapy instructions is further determined based on a comparison between the group physical activity parameter and a user physical activity parameter determined based on the physical activity feature for the user.

13. The system of claim 11, wherein a set of user baseline data for each user in the plurality of users within the group comprises a weight loss goal for said user, and wherein the group physical activity parameter is further calculated based on a percentage weight loss for each user relative to the weight loss goal for said user.

14. The system of claim 11, wherein the physical activity feature for the user is calculated based on a calculated trend relationship in the motion data time series and the weight data time series.

15. The system of claim 14, wherein the calculated trend relationship between the motion data time series and the weight data time series is evaluated on a one-week timescale.

16. The system of claim 11, wherein the set of optimized therapy instructions is calculated for at least one of a set of pathologies, wherein the set of pathologies comprise diabetes and obesity.

17. The system of claim 11, wherein the therapy instruction generation subsystem is further configured to calculate a therapy efficacy parameter based on: a previous set of optimized therapy instructions for the user, the motion data time series, and the weight data time series, wherein the set of optimized therapy instructions is additionally determined based on the therapy efficacy parameter.

18. The system of claim 11, wherein the therapy instruction generation subsystem is further configured to filter the weight data time series, comprising:
calculating a weight trend in the weight data time series; and
removing a datapoint from the weight data time series when a calculated deviation of the datapoint from the weight trend is greater than a threshold value to generate a modified weight data time series;
wherein the physical activity feature is generated based on features extracted from the modified weight data time series.

19. The system of claim 18, wherein the user is clustered into the group based on a similarity between the physical activity feature for the user and the physical activity features for the plurality of users within the group.

20. The system of claim 11, wherein the therapy instruction generation subsystem is further configured to:
generate control instructions for the weight sensor based on the group physical activity parameter, the user baseline data, and the user response data; and
facilitate weight sensor operation according to the control instructions.
